(19) United States
(12) Patent Application Publication
(10) Pub. No.: US 2011/0303218 A1
(43) Pub. Date: Dec. 15, 2011

(54) NASAL NEBULIZER

(76) Inventor: Kambiz Yadidi, Los Angeles, CA (US)

(21) Appl. No.: 13/006,367

(22) Filed: Jan. 13, 2011

Related U.S. Application Data
(60) Provisional application No. 61/353,169, filed on Jun. 9, 2010.

(51) Int. Cl. A61M 11/00

(52) U.S. Cl. 128/200.14

(57) ABSTRACT

A nasal delivery system for and a method of delivering aerosolized medication to the nasal passageway of a patient for treating an ailment in the upper respiratory tract above the trachea, comprising: contacting a nosepiece to a patient's nose, the nosepiece being coupled to a directly to the mesh of the vibrating mist nebulizer via a connecting portion, activating the nebulizer to create aerosolized medication from liquid medication in the chamber, directing the aerosolized medication through the connecting portion to the nosepiece; and dispensing, from apertures in the nosepiece, the aerosolized medication directly through the patient's nose.

### CROSS-REFERENCE TO RELATED APPLICATIONS

This application claims priority benefit under 35 U.S.C. §119(e) from U.S. Provisional Application No. 61/353,169, filed Jun. 9, 2010, the entirety of which is incorporated herein by reference.

### BACKGROUND

1. Field of the Inventions

The present disclosure relates to systems and methods for delivering an aerosolized medication through the airway of a patient.

2. Description of the Related Art

Nebulizers are used for treatment of, for example, cystic fibrosis, asthma, Chronic Obstructive Pulmonary Disease (COPD), and other respiratory diseases. A common technical principal for nebulizers is to either use oxygen, compressed air, or ultrasonic power, as means to break up therapeutic agents into small aerosol droplets, for direct inhalation from the mouthpiece of the device. In therapeutic agents are broken to small aerosol droplets, the mixture of gas and a therapeutic agent particles are directed to the patient's respiratory system for treatment.

### SUMMARY

Described herein are systems and methods for treating an ailment in the upper respiratory tract above the trachea. This area may include, but is not limited to, the nasal cavity, the nasopharynx, the oropharynx, and the larynx.

Embodiments described herein generally relate to medical apparatus and methods. In particular, embodiments relate to systems and methods for delivering aerosolized medication to patients for relieving symptoms associated with ailments in the upper respiratory tract, including, headaches (e.g., migraine headaches, tension-type headaches, cluster headaches), facial pain (e.g., trigeminal neuralgia), allergies (rhinitis, sinusitis, and conjunctivitis), asthma, jaw pain, nervous disorders (e.g., epilepsy, Parkinson's), and other ailments.

Other ailments, which may require treatment include nasal inflammation, specifically rhinitis, which can be allergic or non-allergic, is often associated with infection and prevents normal nasal function. By way of example, allergic and non-allergic inflammation of the nasal airway can typically affect between 10 and 20% of the population, with nasal congestion of the erectile tissues of the nasal concha, lacrimation, secretion of watery mucus, sneezing and itching being the most common symptoms. As will be understood, nasal congestion impedes nasal breathing. Other nasal conditions include nasal polyps, which can arise from the paranasal sinuses, hypertrophic adenoids, secretory otitis media, sinus disease and reduced olfaction.

In the treatment of certain nasal conditions, the topical administration of medicaments is preferable, particularly where the nasal mucosa is the prime pathological pathway, such as in treating or relieving nasal congestion. Medicaments that are commonly topically delivered include decongestants, anti-histamines, cromoglycates, steroids and antibiotics. At present, among the known anti-inflammatory pharmaceuticals, topical steroids have been shown to have an effect on nasal congestion. Topical decongestants have also been suggested for use in relieving nasal congestion. The treatment of hypertrophic adenoids and chronic secretory otitis media using topical decongestants, steroids and anti-microbial agents, although somewhat controversial, has also been proposed. Further, the topical administration of pharmaceuticals has been used to treat or at least relieve symptoms of inflammation in the anterior region of the nasopharynx, the paranasal sinuses and the auditory tubes.

Medicaments can also be systemically delivered through the nasal pathway, the nasal pathway offering a good administration route for the systemic delivery of pharmaceuticals, such as hormones, for example, oxytocin and calcitionin, and analgetics, such as anti-migraine compositions, as the high blood flow and large surface area of the nasal mucosa advantageously provides for rapid systemic uptake.

Nasal delivery can also be advantageous for the administration of medicaments requiring a rapid onset of action, for example, analgetics, anti-emetics, insulin, anti-epileptics, sedatives and hypnotica, and other pharmaceuticals, for example, cardio-vascular drugs. It is envisaged that nasal administration will provide for a fast onset of action, at a rate similar to that of injection and at a rate much faster than that of oral administration. Indeed, for the treatment of many acute conditions, nasal administration is advantageous over oral administration, since gastric stasis can further slow the onset of action following oral administration.

Nasal delivery may also provide an effective delivery route for the administration of proteins and peptides as produced by modern biotechnological techniques. For such substances, the metabolism in the intestines and the first-pass-effect in the liver represent significant obstacles for reliable and cost-efficiency delivery.

Furthermore, it is expected that nasal delivery using the nasal delivery systems and methods of the present invention will prove effective in the treatment of many common neurological diseases, such as Alzheimer's, Parkinson's, psychiatric diseases and intracerebral infections, where not possible using existing techniques. The nasal delivery technique of the present invention allows for delivery to the olfactory region, which region is located in the superior region of the nasal cavities and represents the only region where it is possible to circumvent the blood-to-brain barrier (BBB) and enable communication with the cerebrospinal fluid (CSF) and the brain.

Nasal delivery provides an advantage in that the nosepiece acts to expand the narrowest, anterior part of the nasal cavity and thereby reduces the unwanted high deposition in the anterior region of the nasal cavity. In addition, a bidirectional air flow through the nasal cavities acts to create a positive pressure inside the nasal passages connected in series, which tends to expand and widen narrow and congested areas. Furthermore, the two-point fixation of the nosepiece in the nose provides a much more stable and reproducible positioning of the device and provides a more user-friendly and intuitive nasal delivery method.

Some embodiments of the present invention describe a system of treating an ailment in the upper respiratory tract above the trachea, including the nasal cavity, the nasopharynx, the oropharynx, and the larynx, the system including: a vibrating mesh to aerosolize medication, a nozzle such that the aerosolized medication can be sprayed to the nasal airway of a patient via a connecting portion, a connecting portion extending substantially from an outlet port of the nebulizer to the nosepiece having a lumen therethrough to conduct aerosolized medication to a patient's nasal passageway from the mesh to the nosepiece, and a nosepiece having an aperture for delivering aerosolized medication to a patient's upper respiratory tract directly through a patient's nose.

Some embodiments of the system provide that the pathway from the mesh of the vibrating mist nebulizer to the aperture of the nosepiece is configured to less than 5 cm. Some embodiments of the system provide that the nosepiece comprises two outlet apertures for directing the microdroplets. Some embodiments of the system provide that a nosepiece adapter could be configured to couple the connecting portion with the nebulizer cup or nozzle.

Some embodiments of the system provide that the microdroplets are moved by creation of a pressure gradient within the connecting member by activating the nebulizer. Some embodiments of the system provide that the vibrating mist nebulizer, connecting portion and nosepiece is configured to provide a positive pressure at the aperture of the nosepiece during aerosolization of the vibrating mist nebulizer.

Some embodiments of the system provide that the connecting member has an internal bore with a cross-sectional area that is greater than the outlet area of apertures in the nosepiece. Some embodiments of the system provide that the connecting portion has an internal bore with a diameter of from about 10 mm to about 17.5 mm. Some embodiments of the system provide that the connecting portion comprises an angulated turn. Some embodiments of the system provide that the angulated turn comprises an angle between about 45° and about 135°.

Some embodiments describe a method of treating an ailment in the upper respiratory tract above the trachea, including the nasal cavity, the nasopharynx, the oropharynx, and the larynx, the method including contacting a nosepiece to a patient's nose, the nosepiece being coupled to a vibrating mist nebulizer, the nosepiece being coupled directly to the mesh of the vibrating mist nebulizer via a connecting portion; activating the nebulizer to create aerosolized medication from liquid medication in the chamber; directing the aerosolized medication through the connecting portion to the nosepiece; and dispensing, from apertures in the nosepiece, the aerosolized medication substantially directly through the patient's nose for treating the ailment in the upper respiratory tract.

Some methods provide that the microdroplets are directed substantially only through the patient's nose from the nosepiece. Some methods provide that the nosepiece comprises two outlet apertures for directing the microdroplets. Some methods provide that a nosepiece adapter could be configured to couple the connecting portion with the nebulizer cup or nozzle.

Some methods provide that the pathway from the mesh of the vibrating mist nebulizer to the aperture of the nosepiece is configured to less than 5 cm. Some methods provide that the microdroplets are moved by creation of a pressure gradient within the connecting member by activating the nebulizer. Some methods provide that the vibrating mist nebulizer, connecting portion and nosepiece is configured to provide a positive pressure at the aperture of the nosepiece during aerosolization of the vibrating mist nebulizer.

Some methods provide that the connecting portion has an internal bore with a cross-sectional area that is greater than the outlet area of apertures in the nosepiece. Some methods provide that the connecting portion has an internal bore with a diameter of from about 10 mm to about 17.5 mm. Some methods provide that the connecting portion comprises an angulated turn. Some methods provide that the angulated turn comprises an angle between about 45° and about 135°.

Some methods described herein provide ways for treating an ailment in the upper respiratory tract above the trachea, including the nasal cavity, the nasopharynx, the oropharynx, and the larynx, the method including: directing the aerosolized medication from an outlet port of a nebulizer through a connecting portion to a nosepiece; and dispensing, from outlet apertures in the nosepiece, the aerosolized medication substantially solely through the patient's nose for treating the ailment in the upper respiratory tract.

Some methods further include contacting a patient's nose with the nosepiece such that the outlet apertures are substantially aligned with the patient's nose. Some methods provide that the aerosolized medication is directed substantially only through the patient's nose from the nosepiece. Some methods provide that the nosepiece comprises two outlet apertures for directing the aerosolized medication. Some methods provide that a nosepiece adapter could be configured to couple the connecting portion with the nebulizer cup or nozzle.

Some methods provide that the pathway from the mesh of the vibrating mist nebulizer to the aperture of the nosepiece is configured to less than 5 cm. Some methods provide that the aerosolized medication is moved by creation of a pressure gradient within the connecting portion by activating the nebulizer. Some methods provide that the vibrating mist nebulizer, connecting portion and nosepiece is configured to provide a positive pressure at the aperture of the nosepiece during aerosolization of the vibrating mist nebulizer.

Some methods provide that the connecting portion has an internal bore with a cross-sectional area that is greater than the outlet area of apertures in the nosepiece. Some methods provide that the connecting portion has an internal bore with a diameter of from about 10 mm to about 17.5 mm. Some methods provide that the connecting portion comprises an angulated turn. Some methods provide that the angulated turn comprises an angle between about 45° and about 135°.

### BRIEF DESCRIPTION OF THE DRAWINGS

The accompanying drawings, which are included to provide further understanding of the subject technology and are incorporated in and constitute a part of this specification, illustrate aspects of the subject technology and together with the description serve to explain the principles of the subject technology.

FIG. 1 depicts a nebulizer with a nosepiece and connecting portion in connection with embodiments described herein.

FIG. 2 depicts another view of a nebulizer with a nosepiece and connecting portion in accordance with the embodiments described herein.

FIG. 3 depicts another view of a nebulizer with a nosepiece, connecting portion, and a nosepiece adapter in accordance with embodiments described herein.

FIG. 4 depicts a connecting portion in accordance with embodiments described herein.

FIG. 5 depicts outlet apertures of a nosepiece in accordance with embodiments described herein.

FIG. 6 depicts embodiments of the nosepiece coupled to a connecting portion in connection with embodiments described herein.

### DETAILED DESCRIPTION OF THE INVENTION

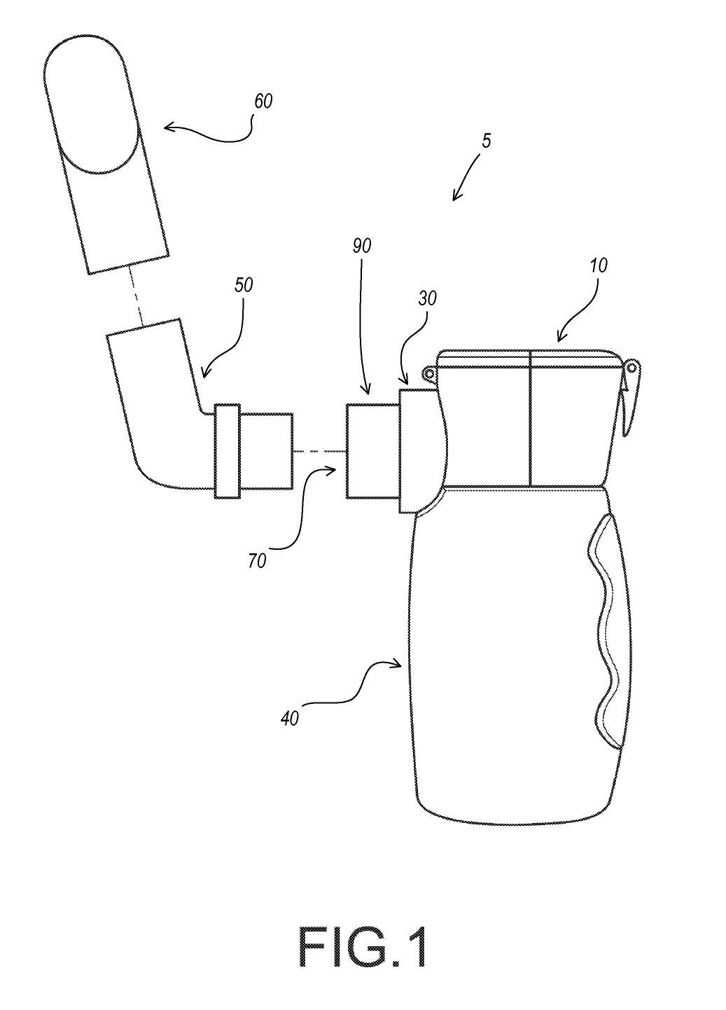

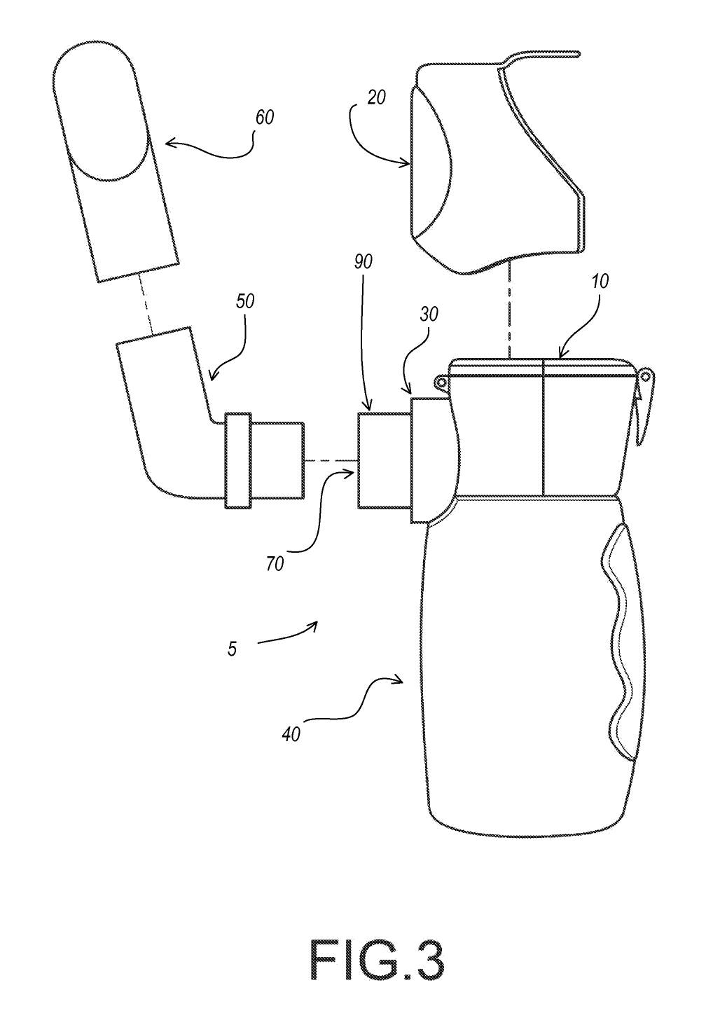

FIGS. 1 to 3, illustrate a nebulizer for delivering an aerosolized medication through the nasal passageway of a patient to a patient's upper respiratory tract in accordance with the embodiments of the present invention. A nasal nebulizer 5 preferably includes a nebulizer cup 10 for containing a liquid, a vibrating mesh 30 installed at a position proximate to an outlet port 90 lateral to the nebulizer cup 10 and interconnected with the nebulizer cup 10 for aerosolizing the liquid supplied in the nebulizer cup 10, and a nosepiece 60 for fitting in the nostrils of the patient which is coupled to the nebulizer via a connecting portion 50.

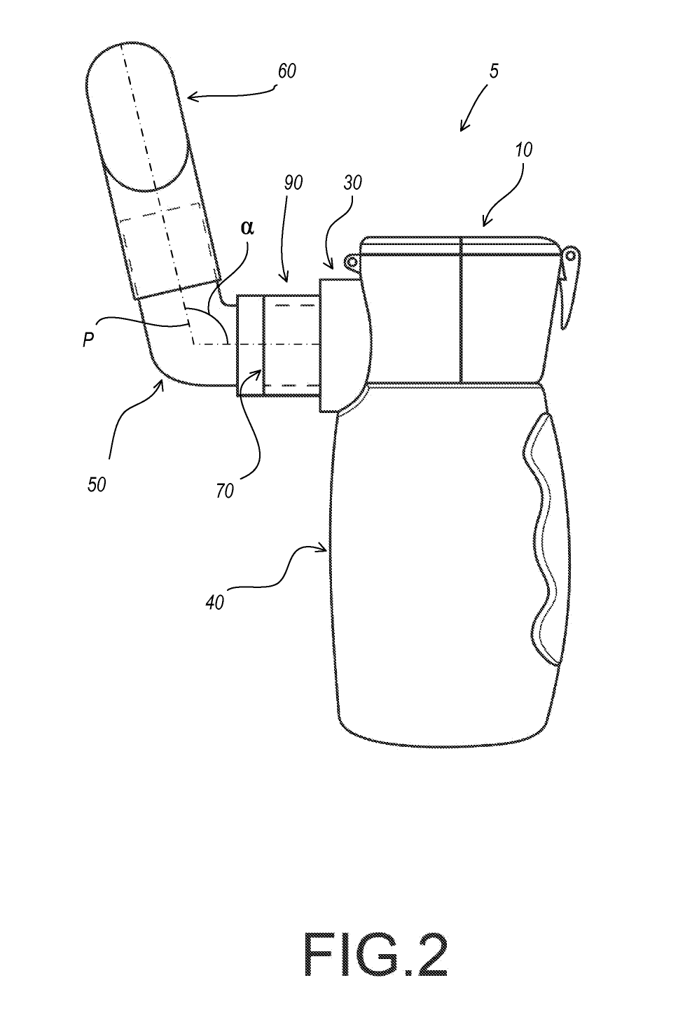

The vibrating mesh 30 is installed at a position proximate to an outlet port 90 lateral to the nebulizer cup 10, which leads to a nozzle 70 such that the liquid can be aerosolized and sprayed from the nozzle 70 to the nasal airway of a patient via a connecting portion 50 extending to the nosepiece 60. In some embodiments, the distance, or path length P (FIG. 2), from the vibrating mesh 30 to the outlet apertures 80 of the nosepiece 60 is less than about 10 cm. In some embodiments, the path length P is less than about 8 cm. In some embodiments, the path length P is less than about 6 cm. In some embodiments, the path length P is less than about 5 cm. In some embodiments, the path length P is less than about 4 cm. The nebulizer cup 10 includes a handle 40 positioned at the bottom of the nebulizer cup 10, and is provided for a patient to hold the nebulizer.

A nosepiece 60 is coupled to the nebulizer via a connecting portion 50 which includes an elbow 53 that directs the aerosolized medication in a different direction than when it leaves the nozzle 70. The nosepiece 60 is configured with outlet apertures 80 for delivering aerosolized medication to a patient's upper respiratory tract directly through a patient's nose. In some embodiments, the cross-sectional area of the outlet apertures 80 in the nosepiece 60 is less than the cross-sectional area of the internal bore of the connecting portion 50. In some embodiments, a nosepiece adapter 20 could be configured to couple the connecting portion 50 extending to the nosepiece 60 with the nebulizer cup 10 or nozzle 70.

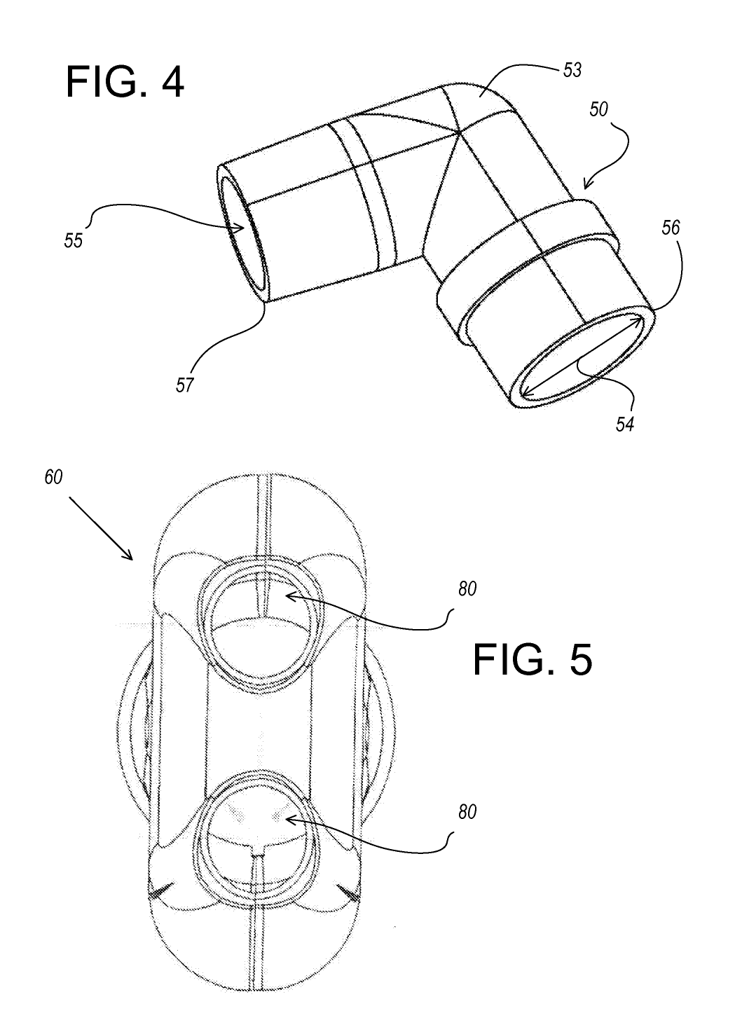

FIG. 4 depicts a connecting portion 50 having an elbow 53 that is directed in a right angle. In some embodiments, the elbow 53 can have an angle α that extends between 45° and 135°. The connecting portion 50 includes a bore extending therethrough, through which aerosolized medication can pass from the nozzle 70 of the nebulizer. In some embodiments, the connecting portion 50 can have an internal bore with a diameter of from about 10 mm to about 17.5 mm. In some embodiments, the inner surface of the connecting portion 50 may contain hydrophobic coating to prevent attachment and loss of any aerosolized medication along the pathway of the connecting portion 50 extending to the nosepiece 60.

The angle α of the elbow 53 can be measured by referencing sections of the connecting portion 50 on both sides of the elbow 53. For example, in some embodiments, the angle α of the elbow 53 is measured by the degree to which a first section of the connecting portion, on a first side of the elbow 53, varies along the length of the first section from a second section of the connecting portion on a second side of the elbow. In some instances, the angle α is taken as the smallest angle between axes defined by the respective first and second sections.

FIG. 5 depicts a top view of the nosepiece 60, having a plurality of outlet apertures 80 that are configured to direct aerosolized medication through the connecting portion 50 and into a patient's nasal passageway. In some embodiments, the nosepiece 60 comprises two outlet apertures 80 for directing the aerosolized medication directly through a patient's nose.

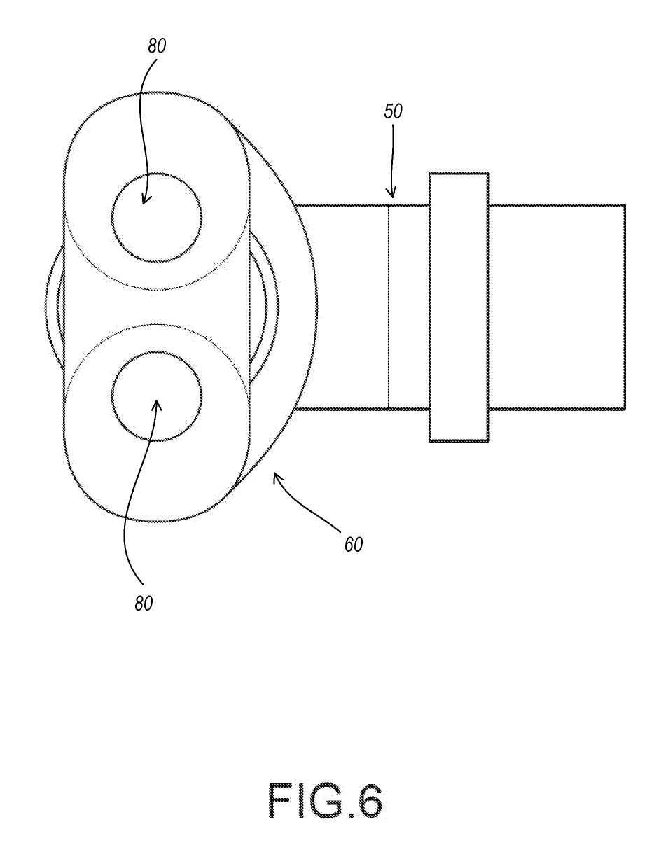

FIG. 6 depicts a top view of the nosepiece 60 coupled to the connecting portion 50 through which aerosolized medication can pass from the nozzle 70 of the nebulizer directly through a patient's nasal passageway. In some embodiments, the nosepiece 60 can be coupled directly to the nebulizer such that aerosolized medication can pass from the nozzle 70 through a patient's nasal airway.

The system can be directed for the treatment of ailments in the upper respiratory tract above the trachea. Treatment of the upper respiratory tract above the trachea is accomplished in a much different fashion than that of treating the ailments of, for example, the lower respiratory tract and/or the lungs. Treatment of the upper respiratory tract can include targeting those areas including the nasal cavity, the nasopharynx, the oropharynx, and the larynx. In these treatments, the system can include a vibrating mesh 30 to aerosolize medication contained in the nebulizer cup 10. The nozzle 70 is preferably adjacent the vibrating mesh 30 and directs the aerosolized medication from the nebulizer. The connecting portion 50 receives the aerosolized medication that is expelled through the vibrating mesh 30 and directs it along a length of the connecting portion 50 to the nosepiece 60.

The nosepiece 60 receives the aerosolized medication from the connecting portion 50 and direct the medication to one of a plurality of outlet apertures 80 for providing the aerosolized medication to the upper respiratory tract of a patient by depositing the medication directly in the patient's nasal passageway. As the patient inhales the aerosolized medication from the nosepiece 60, the aerosolized medication passes through the upper respiratory tract, and is deposited in, for example, the nasal cavity, the nasopharynx, the oropharynx, and the larynx.

In some embodiments, the vibrating mesh aerosolizes the medication and creates a pressure gradient through the connecting portion and the nosepiece. The pressure gradient urges the aerosolized medication through the connecting portion and/or the nosepiece to be ejected from outlet apertures 80 in the nosepiece. Some embodiments provide that during operation of the mesh, a first pressure within the connecting portion adjacent the vibrating mesh is about 10% greater than the ambient pressure. In some embodiments, the first pressure is about 10% greater than a second pressure at the outlet apertures 80. In some embodiments, the first pressure is at least about 15% greater than a second pressure at the outlet apertures 80. In some embodiments, the first pressure is at least about 8% greater than a second pressure at the outlet apertures 80. In some embodiments, the first pressure is at least about 5% greater than a second pressure at the outlet apertures 80.

In some embodiments, the connecting portion 50 has a length from the first end 56 to the second end 57 of less than about 8 cm. In some embodiments, the connecting portion 50 has a length of less than about 6 cm. In some embodiments, the connecting portion 50 has a length of less than about 5 cm. In some embodiments, the connecting portion 50 has a length of less than about 4 cm. In some embodiments, the connecting portion 50 has a length of less than about 2 cm.

The pathway through the connecting portion 50 and then nosepiece 60, from the vibrating mesh 30 to the outlet apertures 80 of the nosepiece 60, has a length of preferably less than about 5 cm. In some embodiments, the pathway has a length of less than about 4 cm. Some embodiments provide that the length of the pathway from the vibrating mesh 30 to the outlet apertures 80 of the nosepiece 60 is less than about 3 cm. In certain embodiments, the pathway has a length of less than about 2 cm. In some embodiments, a shorter length of the pathway is used to, for example, decreased the amount of aerosolized medication that is lost to the interior surfaces of the connecting portion 50 and nosepiece 60 and to increase the amount of positive pressure provided by the aerosolized medication at the outlet apertures 80. Some embodiments can provide a modified nosepiece 60 that connects directly to the nozzle 70 of the nebulizer 5 (not shown). This can reduce even further the distance from the vibrating mesh 30 to the outlet apertures 80. In such embodiments, the pathway from the vibrating mesh 30 to the outlet apertures 80 can be about 1.5 cm or less.

The aerosolized medication move from the vibrating mesh 30 to the outlet apertures 80 by a pressure gradient that is created when the vibrating mesh 30 is operated. The pressure gradient preferably results in a positive pressure that ejects aerosolized medication from the outlet apertures 80 of the nosepiece 60.

The connecting portion 50 preferably has an internal bore 55 with a cross-sectional area that is greater than a combined cross sectional area of the outlet apertures 80. This configuration can assist in providing a positive pressure of the aerosolized medication at the outlet apertures 80. In some embodiments, the internal bore 55 of the connecting portion 50 has a cross-sectional dimension of from about 10 mm to about 17.5 mm. In some embodiments, the cross-sectional dimension is from about 12 mm to about 15 mm. In some embodiments, the cross-sectional dimension of the internal bore 55 of the connecting portion 50 is between about 13 mm and about 15 mm. Some embodiments provide that the cross-sectional dimension of the internal bore 55 can be greater than about 17.5 mm. In some embodiments, a cross-sectional dimension of the internal bore 55 can be less than about 10 mm.

In some embodiments, the internal bore 55 has a cross-sectional dimension 54 that decreases along the path length of the connecting portion 50 from a first end 56 to a second end 57. In some instances, the internal bore 55 can begin at the first end 56 with a cross-section dimension of from about 10 mm to about 17.5 mm and can taper gradually along its length to the second end 57, which can have a cross-sectional dimension of from about or less than about 10 mm to about 15 mm. In some embodiments, this tapering of the internal bore 55 can assist with maintaining a pressure gradient within the connecting portion 50 to assist in providing a positive pressure from the outlet apertures 80.

In some embodiments, the internal bore 55 cross-section dimension decreases along the path length of the connecting portion 50 by about 15%. In some embodiments, the internal bore 55 cross-section dimension decreases along the path length of the connecting portion 50 by between about 10% and about 20%. In some embodiments, the internal bore 55 cross-section dimension decreases along the path length of the connecting portion 50 by between about 15% and about 25%.

In some embodiments, the internal bore 55 cross-section dimension decreases along only a portion of the path length of the connecting portion 50. For example, in some embodiments, the cross-section dimension decreases along only 50% along the path length of the connecting portion 50. In some embodiments, the internal bore 55 decreases between about 25% and about 45% of the path length within the connecting portion 50. In some embodiments the about 25% to about 45% of the path length through which the internal bore 55 decreases is adjacent to the second end 57 of the connecting portion 50. In some embodiments the about 25% to about 45% of the path length through which the internal bore 55 decreases is adjacent to the first end 56 of the connecting portion 50.

In some embodiments, the internal bore 55 decreases between about 35% and about 75% of the path length within the connecting portion 50. In some embodiments the about 35% to about 75% of the path length through which the internal bore 55 decreases begins at a location adjacent to the second end 57 of the connecting portion 50. In some embodiments the about 35% to about 75% of the path length through which the internal bore 55 decreases begins at a location adjacent to the first end 56 of the connecting portion 50.

The connecting portion 50 can also include an angulated turn along its length. In some embodiments, the angulated turn has an angle α between about 45° and about 135°. In some embodiments, the angulated turn has an angle between about 90° and about 140°. In some embodiments, the angulated turn has an angle between about 95° and about 135°. In some embodiments, the angulated turn has an angle between about 100° and about 130°. In some embodiments, the angulated turn has an angle between about 105° and about 115°. In some embodiments, the angulated turn has an angle of about 110°. In some embodiments, the angulated turn has an angle of about 120°. In some embodiments, the angulated turn has an angle between about 120° and about 180°. Some embodiments can provide a connecting portion 50 with an angulated turn along its length having an angle less than about 45°. Some embodiments can provide a connecting portion 50 with an angulated turn along its length having an angle greater than about 135°.

Methods of treating an element in the upper respiratory tract can include providing a vibrating mist nebulizer 5, as discussed above, and contacting the nosepiece to a patient's nose. With the nosepiece 60 in contact with the patient's nose, and the nosepiece 60 being coupled to a vibrating mist nebulizer 5 by a connection portion 50, the method further includes activating the nebulizer 5 to create aerosolized medication from liquid medication in a chamber, or nebulizer cup 10, and directing the aerosolized medication through the connecting portion 50 to the nosepiece 60. The method further includes dispensing, from apertures 80 in the nosepiece 60, the aerosolized medication substantially directly through the patient's nose for treating the ailment in the upper respiratory tract.

For treatment of ailments in the upper respiratory tract, the microdroplets are preferably directed substantially only through the patient's nose from the nosepiece 60. Some methods provide that the nosepiece 60 comprises two outlet apertures 80 for directing the microdroplets to each nostril of the patient. Some methods include using a nosepiece adapter 20 to couple the connecting portion 50 with the nebulizer cup 10 or nozzle 70 and to reduce or prevent loss of aerosolized medication through improperly assembled nebulizers 5. For example, in some embodiments, the nosepiece adapter 20 cannot be properly positioned on the nebulizer 5 unless the connecting portion 50, or the nosepiece 60, is properly coupled to the nebulizer 5.

Some embodiments may provide that the nebulizer 5 will not properly operate unless the nosepiece adapter 20 is properly positioned on the nebulizer 5. For example, in some embodiments, the nebulizer 5 can include a safety switch that is depressed when the nosepiece adapter 20 is properly positioned on the nebulizer 5. In such embodiments, if the safety switch is not depressed, the nebulizer 5 will not operate when an activation button is depressed or when other actuating mechanisms are activated.

The nebulizer 5 can have a feedback system that will prevent the nebulizer 5 from operating unless the safety switch indicates that the nosepiece adapter 20 is properly positioned. When the nosepiece adapter 20 is properly positioned, the safety switch is depressed, and the feedback system will permit activation of the nebulizer 5. When the activation button is depressed or when other actuating mechanisms are activated.

While the invention has been described by way of examples and in terms of preferred embodiments, it is to be understood that the invention is not limited thereto. To the contrary, it is intended to cover various modifications and similar arrangements and procedures, and the scope of the appended claims therefore should be accorded the broadest interpretation so as to encompass all such modifications and similar arrangements and procedures.

What is claimed is:

1. A system, for delivering medication to a patient's upper respiratory tract above the trachea, comprising:
a mesh that vibrates to aerosolize medication;
a nozzle, in communication with the mesh, such that the aerosolized medication from the mesh passes through the nozzle;
a connecting portion coupled to the nozzle and in communication with the mesh, the connecting portion having a lumen to conduct aerosolized medication to a patient's nasal passageway from the mesh, the connecting portion having a length, from a first end to a second end, of less than about 6 cm, and the connecting portion having an elbow that separates a first section of the connecting portion and a second section of the connecting portion, the elbow having an angle, such that the second section extends, relative to the first section, at an angle of between about 45° and about 135°; and
a nosepiece coupled to the connecting portion, the nosepiece having an aperture at a distal end, that delivers aerosolized medication to a patient's upper respiratory tract directly through a patient's nose from the aperture.

2. The system of claim 1, wherein a pathway from the mesh to the aperture of the nosepiece is less than about 5 cm.

3. The system of claim 1, wherein the angle is between about 90° and about 135°.

4. The system of claim 1, wherein the nosepiece comprises two outlet apertures for directing the medication.

5. The system of claim 1, further comprising a nosepiece adapter configured to couple the connecting portion extending to the nosepiece with the nebulizer.

6. The system of claim 1, wherein the connecting portion has an internal bore with a cross-sectional area that is greater than the outlet area of apertures in the nosepiece.

7. The system of claim 1, wherein the connecting portion has an internal bore with a diameter of from about 10 mm to about 17.5 mm.

8. The system of claim 1, wherein the connecting portion has an internal bore with a cross-sectional dimension that decreases from the mesh to the nosepiece.

9. A method for treating a patient's upper respiratory tract above the trachea, including the nasal cavity, the nasopharynx, the oropharynx, and the larynx, the method comprising:
contacting a nosepiece to a patient's nose, the nosepiece being coupled to a vibrating mist nebulizer, the nosepiece being coupled directly to the mesh via a connecting portion extending substantially from an outlet port of the nebulizer;
activating the nebulizer to create aerosolized medication from liquid medication in the chamber;
directing the aerosolized medication through the connecting portion to the nosepiece, the connecting portion having an angulation of between about 90° and about 135°; and
dispensing, from apertures in the nosepiece, the aerosolized medication directly through the patient's nose for treating the ailment in the upper respiratory tract;
wherein a path length of the aerosolized medication from the mesh to the aperture of the nosepiece is less than about 10 cm.

10. The method of claim 9, wherein the aerosolized medication is directed through the nosepiece from the mesh to substantially only the patient's nose.

11. The method of claim 9, wherein the length from the mesh to the aperture of the nosepiece is less than cm.

12. The method of claim 9, wherein the nosepiece comprises two outlet apertures for directing the medication.

13. The method of claim 9, further comprising coupling the connecting portion with the nebulizer with a nosepiece adapter that mechanically locks the connecting portion with the nebulizer.

14. The method of claim 9, wherein the connecting portion has an internal bore with a cross-sectional area that is greater than the outlet area of apertures in the nosepiece.

15. The method of claim 9, wherein the path length is less than about 8 cm.

16. The method of claim 9, wherein the path length is less than about 6 cm.

17. The method of claim 9, wherein the connecting portion has an internal bore with a diameter of from about 10 mm to about 17.5 mm.

18. A system for delivering medicated aerosolized droplets to a patient's upper respiratory tract above the trachea, including the nasal cavity, the nasopharynx, the oropharynx, and the larynx, the system comprising:
a mesh that vibrates to aerosolize medication;
a nozzle, in communication with the mesh, such that the aerosolized medication from the mesh passes through the nozzle;
a connecting portion coupled to the nozzle and in communication with the mesh, the connecting portion having a lumen to conduct aerosolized medication to a patient's nasal passageway from the mesh; and
a nosepiece coupled to the connecting portion, the nosepiece having an aperture for delivering aerosolized medication to a patient's upper respiratory tract directly through a patient's nose;
wherein upon vibration of mesh, aerosolized medication is ejected from the mesh into the nozzle and connecting portion such that a first pressure at the mesh is at least about 10% greater than a second pressure at the nosepiece aperture, such that a pressure gradient created from the connecting portion to the nosepiece urges the aerosolized medication through the connecting portion and nosepiece, and the aerosolized medication is expelled from the nosepiece aperture by the pressure gradient.

19. The system of claim 18, wherein a pathway from the mesh to the aperture of the nosepiece is configured to less than 5 cm.

20. The system of claim 18, wherein the nosepiece comprises two outlet apertures for directing the medication.

21. The system of claim 18, wherein a nosepiece adapter is configured to couple the connecting portion extending to the nosepiece with the nebulizer.

22. The system of claim 18, wherein the connecting portion has an internal bore with a cross-sectional area that is greater than the outlet area of apertures in the nosepiece.

23. The system of claim 18, wherein the connecting portion has an internal bore with a diameter of from about 10 mm to about 17.5 mm.

24. The system of claim 18, wherein the connecting portion comprises an angulation between about 45° and about 135°.

25. A method for delivering medicated aerosolized droplets to a patient's upper respiratory tract above the trachea, including the nasal cavity, the nasopharynx, the oropharynx, and the larynx, the method comprising:
directing the aerosolized medication from an outlet port of a nebulizer through a connecting portion to a nosepiece; and
dispensing, from outlet apertures in the nosepiece, the aerosolized medication substantially solely through the patient's nose by the creation of a pressure gradient from the mesh to the nosepiece;
wherein aerosolized medication is ejected from the mesh into the nozzle and connecting portion such that a first pressure of the aerosolized medication in the connecting portion adjacent the mesh is at least about 10% greater than a second pressure at the outlet apertures.

26. The method of claim 25, further comprising contacting a patient's nose with the nosepiece such that the outlet apertures are substantially aligned with the patient's nose.

27. The method of claim 25, wherein the aerosolized medication is expelled from the nosepiece only through a nosepiece aperture.

28. The method of claim 25, wherein the nosepiece comprises two outlet apertures for directing the medication.

29. The method of claim 25, wherein a nosepiece adapter is configured to couple the connecting portion extending to the nosepiece with the nebulizer.

30. The method of claim 25, wherein a pathway from the mesh to the aperture of the nosepiece is configured to less than 5 cm.

31. The method of claim 25, wherein the vibrating mesh, connecting portion and nosepiece are configured to provide a positive pressure at the aperture during aerosolization of the vibrating mesh.

32. The method of claim 25, wherein the connecting portion has an internal bore with a cross-sectional area that is greater than the outlet area of apertures in the nosepiece.

33. The method of claim 25, wherein the diameter of the connecting portion is selected to produce positive pressure to urge the aerosolized medication therethrough.

34. The method of claim 25, wherein the connecting portion has an internal bore with a diameter of from about 10 mm to about 17.5 mm.

35. The method of claim 25, wherein the connecting portion comprises an angulated turn.

36. The method of claim 35, wherein the angulated turn comprises an angle between about 45° and about 135°.
